(19) United States
(12) Patent Application Publication
(10) Pub. No.: US 2006/0228694 A1
(43) Pub. Date: Oct. 12, 2006

(54) Methods to individualize combination therapy

(76) Inventors: Andrew S. Janoff, Yardley, PA (US); Lawrence Mayer, North Vancouver (CA)

Correspondence Address: MORRISON & FOERSTER LLP, 12531 HIGH BLUFF DRIVE, SUITE 100, SAN DIEGO, CA, 92130-2040, US

(21) Appl. No.: 10/551,374

(22) PCT Filed: Apr. 2, 2004
(86) PCT No.: PCT/US04/10388

§ 371 Date: Mar. 30, 2006

Related U.S. Application Data
(63) Continuation-in-part of application No. 60/496,180, filed on Aug. 18, 2003.
(60) Provisional application No. 60/460,223, filed on Apr. 2, 2003.
(60) Provisional application No. 60/495,394, filed on Aug. 15, 2003.

(51) Int. Cl. A61K 38/17; C12Q 1/00; C12Q 1/68

(52) U.S. Cl. 435/4; 514/2; 435/6

(57) ABSTRACT

Compositions which comprise delivery vehicles having stably associated therewith predetermined, patient-specific combinations of two or more agents, such as antineoplastic agents, are useful in achieving optimal therapeutic effects on a patient-by-patient basis when combinations of drugs are administered.

This application claims benefits under 35 USC 119(e) of U.S. Ser. No. 60/460,223 filed 2 Apr. 2003, 60/495,394 filed 15 Aug. 2003, and 60/496,180 filed 18 Aug., 2003. The contents of these applications are incorporated by reference.

### TECHNICAL FIELD

The invention relates to compositions and methods for administering improved therapies utilizing combinations of therapeutic agents. More particularly, the invention concerns methods to identify and generate combination therapies optimized for individual patients.

### BACKGROUND

It is well understood that the metabolic response to pharmaceuticals in humans is far from uniform. In addition to what might be considered simply side effects (such as allergies to various antibiotics), drugs that are designed to treat more chronic conditions, such as arthritis, cancer, inflammation, and the like, do not elicit the same responses in all individuals. The recognition of this heterogeneity in the patient population has inspired the concept of pharmacogenomics where, a genomic fingerprint of an individual is used in combination with certain historical data to design appropriate treatments. A more direct approach to tailoring treatment to an individual patient is illustrated by the practice surrounding the administration of Herceptin® in the treatment of breast tumors; only individuals whose tumors display the HER2 receptor with which Herceptin® interacts are offered this treatment.

Unfortunately, the progression of many life-threatening diseases such as cancer, AIDS, infectious diseases, immune disorders and cardiovascular disorders is influenced by multiple molecular mechanisms. Due to this complexity, achieving effective treatments using a single agent has been met with limited success. Thus, combinations of agents have often been used to combat disease, specifically in the treatment of cancers. It appears that there is a strong correlation between the number of agents administered and cure rates for cancers such as acute lymphocytic leukemia and histiocytic lymphoma (Frei, et al., Clin. Cancer Res. (1998) 4:2027-2037; Todd, et al., J. Clin. Oncol. (1984) 2:986-993).

Although the use of combination therapies, in particular in the treatment of tumors, is widely practiced, it is met with very much less than a perfect record of success. One of the reasons for this lack of success in view of the present applicants is the failure to individualize treatments, in accordance with the particular characteristics of the individual being treated.

There are a growing number of indicators suggesting that cancer is the result of a series of genetic alterations that regulate the behavior of primitive normal cells. Moreover, it appears that the development of a malignant phenotype by these cells may be achieved through changes in the expression of a variety of genes. Cancer, therefore, needs to be understood as a genetically heterogeneous disease, and a more refined knowledge of the key changes in individual tumors will be required to develop more specific and individualized therapeutic procedures. As indicated above, breast cancer patients have already benefited from the use of a rationally based, molecularly targeted therapy following the initial discovery of the HER-2/neu oncogene and the hallmark proof of concept study of targeted therapy with Herceptin® in HER-2/neu overexpressing tumors.

The still poorly understood heterogeneity of human cancers is highlighted in two recent reports on breast cancer biopsies obtained from individual breast cancer patients. In a seminal study, Perou, et al., used cDNA microarray data to demonstrate that human breast cancers could be classified into several biologically distinct subtypes according to their gene expression profiles: basal epithelia-like; HER2/neu-overexpressing; and a normal breast like group; each of which could be similarly divided into unique subgroups (Nature (2000) Aug. 17, 406(6797): 747-752). Recently, van 't Veer, et al., further showed that assessment of the gene expression ‘signature’ of patients' breast tumors was predictive of those with node negative disease who would benefit from adjuvant chemotherapy (Nature (2002) Jan. 31, 415(6871): 530-536). These findings point to the reality and value of using biomarker assays (to determine one's molecular phenotype), in combination with current pathological assessment criteria, to significantly improve the use of existing therapeutic modalities. Globally these studies will facilitate the development of a robust molecular taxonomy of cancer that may largely replace current pathological approaches in the next decade.

As suggested in the above example, tumor heterogeneity can be observed in patients with cancers arising from similar cell types. Therefore, chemotherapeutic agents designed to combat a particular cancer will differentially affect patients with this cancer. Due to this, finding a single drug combination product designed to effect treatment of a broad patient population may actually only be effective in a defined subset of patients. This problem is confounded by the fact that drugs are distributed and metabolized at different rates and therefore administering a combination of free drugs at a set ratio will likely result in a rapid and uncontrolled change of the drug/drug ratio. The problem becomes even more difficult as each person being treated will distribute and metabolize the administered drugs at different rates and therefore controlling the pharmacokinetics of each drug (and thus their ratio) becomes an issue on a patient-by-patient basis.

Controlling the drug ratio is critical as the effects of combinations of drugs can be enhanced when the ratio in which they are supplied is additive or synergistic. Synergistic combinations of agents have also been shown to reduce toxicity due to lower dose requirements, to increase cancer cure rates (Barriere, et al., Pharmacotherapy (1992) 12:397-402; Schimpff, Support Care Cancer (1993) 1:5-18), and to reduce the spread of multi-resistant strains of microorganisms (Shlaes, et al., Clin. Infect. Dis. (1993) 17:S527-S536). By choosing agents with different mechanisms of action, multiple sites in biochemical pathways can be attacked thus resulting in synergy (Shah and Schwartz, Clin. Cancer Res. (2001) 7:2168-2181). Combinations such as L-canavanine and 5-fluorouracil (5-FU) have been reported to exhibit greater antineoplastic activity in rat colon tumor models than the combined effects of either drug alone (Swaffar, et al., Anti-Cancer Drugs (1995) 6:586-593). Cisplatin and etoposide display synergy in combating the growth of a human small-cell lung cancer cell line, SBC-3 (Kanzawa, et al., Int. J. Cancer (1997) 71(3):311-319).

In the foregoing studies, the importance of the ratio of the components for synergy was recognized. For example, 5-fluorouracil and L-canavanine were found to be synergistic at a mole ratio of 1:1, but antagonistic at a ratio of 5:1; cisplatin and carboplatin showed a synergistic effect at an area under the curve (AUC) ratio of 13:1 but an antagonistic effect at 19:5.

Other drug combinations have been shown to display synergistic interactions although the dependency of the interaction on the combination ratio was not described. This list is quite extensive and is composed mainly of reports of in vitro cultures, although occasionally in vivo studies are included. In addition to the multiplicity of reports, numerous combinations have been shown to be efficacious in the clinic.

Despite the advantages associated with the use of synergistic drug combinations, there are various drawbacks that limit their therapeutic use. For instance, synergy often depends on various factors such as the duration of drug exposure and the sequence of administration (Bonner and Kozelsky, Cancer Chemother. Pharmacol. (1990) 39:109-112). Studies using ethyl deshydroxy-sparsomycin in combination with cisplatin show that synergy is influenced by the combination ratios, the duration of treatment and the sequence of treatment (Hofs, et al., Anticancer Drugs (1994) 5:35-42).

It is known that in order for synergy to be exhibited by a combination of agents, these agents must be present in defined ratios. The same combination of drugs may be antagonistic at some ratios, synergistic at others, and additive at still others. Furthermore, whether a particular ratio is synergistic, additive, or antagonistic is concentration dependent. Some ratios are antagonistic at particular drug concentrations and non-antagonistic at others. Thus it is desirable that the ratio be selected at which non-antagonistic results are obtained over a wide concentration range, since the concentration of drugs reaching the target may be different from that administered.

As described below, the problem of maintaining a desired ratio (i.e., non-antagonistic) is solved by the recognition that when therapeutic agents are encapsulated in delivery vehicles, such as liposomes, the delivery vehicles determine the pharmacokinetics and thus agents that are encapsulated will show a more similar biodistribution in all patients. Therefore, the response to treatment will likely be improved as judged by an individual's response (due to optimization of synergistic drug effects) and significantly more patients should exhibit a response to the treatment (due to better control over the pharmacokinetics of the associated synergistic drugs within delivery systems).

The heterogeneity among humans can be accommodated by identifying non-antagonistic ratios of drugs for individual patients and then creating a dosage optimized for each patient. This is necessary because individual patients may differentially respond to different drug combinations and thus a combination specific for each patient need be identified. While non-antagonistic ratios of drugs are commonly identified using only in vitro cultured cells, these ratios may not be equally therapeutically active if administered to all subjects. In order to circumvent these problems, the present invention details a method for selecting drug combinations and non-antagonistic ratios effective in the treatment of molecularly defined cancers to thus generate patient-specific ratios. Molecular taxonomy of cancers will identify better-defined patient populations, leading to selection of ratios better suited for individual patients. Therefore, the invention details a method for matching the molecular phenotype (expression profiling of multiple disease-related biological markers, such as the HER2 receptor) of cultured cells, that have or will undergo drug screening, with the molecular phenotype of particular diseased patients (by using cells from a patient's biopsy or blood) in order to identify drug combinations that provide optimal non-antagonistic effects for individual patients prior to generating a specific drug therapy tailored to each patient. Once information from the patient's tumor has been assessed and the ratio has been selected, generating the patient-specific pharmaceutical preparation for administration can be easily done either in a laboratory or hospital by combining the appropriate amounts of encapsulated drugs at the desired patient-specific ratio.

### DISCLOSURE OF THE INVENTION

The present disclosure relates to methods for preparing pharmaceutical compositions or preparations that individualize combination drug therapies to patients in need thereof using delivery vehicle compositions that encapsulate two or more therapeutic agents. The disclosed methods not only identify an optimal patient-specific ratio of the effective therapeutic agents, but also ensure that the therapeutic agent combination is delivered to the target site at the desired ratio. Typically the desired ratio of therapeutic agents, upon administration, provides a synergistic or additive therapeutic effect as compared to the administration of either therapeutic agent alone. In a preferred embodiment, the therapeutic agents are antitumor drugs, although a variety of other agents are contemplated for use in the invention methods.

Cancer is a genetically heterogeneous disease. To prepare pharmaceutical preparations that are individualized to a particular patient's cancer, diseased tissue or cells are obtained from the patient for molecular characterization or phenotyping. “Patient's cells” refers to cells that have been harvested through mechanical and chemical means, if necessary, from the diseased tissue or blood of an individual patient. The molecular characterization of a patient's cells involves determining the expression pattern of various biomarkers that are used to identify and classify samples. After the sample is obtained from the diseased patient, for example through biopsy or blood/tissue sampling, the diseased cells are subjected to molecular phenotyping to obtain a genomic/proteomic fingerprint designed to classify the cancer into biologically distinct subtypes.

Once a patient's cancer has been classified into a biologically distinct subtype, it can be matched against the molecular phenotype from a variety of cultured cell lines. There are a number of libraries and databases of historical data on biological markers from cultured cell lines that can be easily accessed for comparison. An example of a library is the NCI 60 cell line library. The molecular phenotype of the cultured cells can also be determined at or near the time of comparison to the patient's cells. The cultured cell lines are then used as experimental surrogates for the patient's diseased cells and permit the preparation of a pharmaceutical composition that is individualized to a particular patient's cancer.

The individualized pharmaceutical composition is prepared based on the reactivity of the cultured cell lines to a variety of therapeutic reagents. Multiple combinations of therapeutic agents are provided to the cultured cell lines at multiple ratios to identify those combinations of therapeutic agents that act synergistically against the cultured cell lines. In this way, an optimal non-antagonistic ratio of therapeutic agents can then be identified. For example, a 1:5 ratio of therapeutic agents A and B may have been identified as providing synergistic effects against a particular cultured cell line. A patient presenting a neoplasm submits a sample and it is determined that the patient's neoplasm presents biomarkers similar to that of the cultured cell line. This similarity allows one to classify the patient's neoplasm and the cultured cell line in the same biologically distinct subtype. Because a 1:5 ratio of therapeutic agents A and B acted optimally against the cultured cells with molecular phenotypes that matched those of the patient's diseased cells, this ratio of therapeutic agents is prepared for administration to the individual. Thus, the details of this patient-specific ratio of therapeutic agents are used to prepare a pharmaceutical preparation individualized for the patient in question. (See FIG. 4.)

Whether prepared for administration in the same delivery vehicle or in separate delivery vehicles, the final result is the preparation of an individualized pharmaceutical preparation. The individualized pharmaceutical preparation contains a therapeutic agent combination formulated at a specific ratio that is unique to an individual patient. The delivery vehicle used to package the therapeutic agents are selected to maintain the patient specific ratio using coordinated pharmacokinetics after in vivo administration. By “coordinated pharmacokinetics” is meant that the delivery vehicles assure maintenance of the ratio of the therapeutic agents administered. Maintenance of the ratio occurs whether the therapeutic agents are provided within the same delivery vehicle or in separate delivery vehicles. Once generated, the pharmaceutical preparation may then be administered to the patient, stored for future administration, or duplicated for multiple administrations.

In summary, therapeutic agents are provided to a patient via one or more delivery vehicles such that the therapeutic agents are released at ratios that yield synergistic or additive (that is, non-antagonistic) results in an individual patient. The patient-specific ratio of therapeutic agents is selected based on the ability of the combination to exhibit synergistic or additive effects on cultured cells. The cultured cells used to assay the therapeutic agent combinations typically display a molecular phenotype similar or identical to that of diseased cells from the patient in question.

Thus, in one aspect, the invention provides a patient-specific delivery vehicle composition for parenteral administration comprising two or more therapeutic agents encapsulated in the vehicle composition at a patient-specific ratio that is synergistic or additive.

In another aspect, the invention provides a method to identify a patient-specific non-antagonistic ratio of agents. The patient-specific non-antagonistic ratio is determined by harvesting cells or blood from the diseased patient in order to characterize their molecular phenotype, and then identifying combinations of therapeutic agents that exhibit synergistic or additive effects (preferably using a combination index (CI)) to cultured cells which express a molecular phenotype similar or identical to the cells harvested from the patient in question. Preferably the CI is synergistic over a wide concentration range. Preferred agents are anticancer agents. Any method which results in determination of a ratio of agents which maintains a non-antagonistic effect on cells may be used. Any method which results in determination of the molecular phenotype of cultured or harvested cells may be used.

More particularly, the invention relates to a method to identify a patient-specific composition which comprises delivery vehicles, said delivery vehicles having encapsulated therein at least a first and a second therapeutic agent in a mole ratio of the first agent to the second agent which exhibits a non-antagonistic biologic effect to cultured cells (which display a molecular phenotype similar or identical to cells harvested from the diseased patient) over at least 5% of such concentration range where greater than 1% of said cultured cells are affected (Fraction affected (fₐ)>0.01) or to a composition which comprises delivery vehicles, said delivery vehicles having encapsulated therein at least a first and a second therapeutic agent in a mole ratio of the first agent to the second agent which exhibits a non-antagonistic cytotoxic effect or cytostatic effect to said cultured cells wherein said agents are antineoplastic agents. “Cultured cells,” refer to at least one cell culture or cell line which is appropriate for testing the desired biological effect. For example, if the therapeutic agent is an antineoplastic agent, a “relevant” cell would be a cell line identified by the Developmental Therapeutics Program (DTP) of the National Cancer Institute (NCI)/National Institutes of Health (NIH) as useful in their anticancer drug discovery program. Currently the DTP screen utilizes 60 different human tumor cell lines. The desired activity on at least one of such cell lines would need to be demonstrated.

“Patient's cells” or “cells harvested from the diseased patient,” refers to a collection of cells that have been harvested in vitro, through known mechanical or chemical techniques if necessary, from a sample of the diseased tissue or blood taken from a single patient after removal of the diseased tissue or blood from said patient. For example, if the disease being treated is cancer, “patient's cells” or “cells harvested from the diseased tissue or blood of an individual patient,” would be a collection of cells that have been harvested in the laboratory from a patient's cancerous tissue or blood after removal of the whole tumor, tumor biopsy or blood sample from the patient by a qualified individual. As previously mentioned, by “patient-specific” synergistic, additive or non-antagonistic ratio, the applicants refer to those ratios that have been identified for an individual patient by matching the patient's molecular phenotype with the molecular phenotype of at least one cultured cell line and then choosing, for the patient, the ratio of therapeutic agents which optimally effects the matched cultured cells in in vitro screening analysis.

Characterization of the molecular or cellular phenotype of patient's cells or cultured cells can be carried out by known techniques in the art, including diagnostic immunohistochemistry using tissue microarrays as described by Liu, et al. (Amer. J. of Pathology (2002) 161(5):1557-1565). Typical cancer-specific markers that may be screened for are: Epithelial markers (e.g., epithelial membrane antigen); keratin, CytoKeratin Subset Markers (e.g., cam5.2 (cytokeratin 8/18), cytokeratin (ck) 5-6, ck7, ck19, ck20, and ck22); Adenocarcinoma Markers (e.g., carcinoembryonic antigen, CEA polyclonal-Carcinoembryonic antigen and B72-3); Carcinoma Subset Markers (e.g., thyroid transcription factor 1, synaptophysin, placental alkaline phosphatase and estrogen receptor); Melanoma Markers (e.g., S-100 (polyclonal) and melan-a); Sarcoma Markers (e.g., vimentin and kp 1 (with CD68)); Sarcoma Subset Markers (e.g., o13 and c-kit); and Oncogene Markers (e.g., bcl-2 and p53).

As further described below, in a preferred embodiment, in designing an appropriate combination in accordance with the method described above, the non-antagonistic patient-specific ratios are selected as those in which the matched cultured cells demonstrate a combination index (CI) of ≦1.1, as defined by relevant in vitro screening analysis.

In another aspect, the invention is directed to a method to prepare a patient-specific therapeutic composition comprising delivery vehicles, said delivery vehicles containing a ratio of at least two therapeutic agents which is non-antagonistic to cultured cells (which have a molecular phenotype matching that of said patient), which method comprises providing a panel of at least two therapeutic agents wherein the panel comprises at least one, but preferably a multiplicity of ratios of said agents, testing the ability of the members of the panel to exert a biological effect on cultured cells, selecting a member of the panel wherein the ratio provides a synergistic or additive effect on said cells; and either 1) encapsulating (i.e., stably associating) the ratio of agents represented by the successful member of the panel into drug delivery vehicles or, 2) combining appropriate amounts of pre-formulated vehicle-encapsulated therapeutic agents at the ratio of agents represented by the successful member of the panel into pharmaceutical preparations suitable for administration to the patient whose cellular/molecular phenotype were matched against those of said cultured cells. For example, if in vitro screening on cultured cells (with phenotypes matching those of an individual patient) determined that a 1:5 ratio of drugs A-to-B provided optimal non-antagonistic effects to said cultured cells, a laboratory technician, pharmacist, physician or other authorized individual could combine one part of a pharmaceutical preparation of drug A (pre-formulated to be stably associated with a drug delivery vehicle) with five parts of a pharmaceutical preparation of drug B (pre-formulated to be stably associated with a drug delivery vehicle) to generate a patient-specific combination treatment which could be subsequently administered to said patient using standard procedures; or, if a pharmaceutical preparation has already been made with drugs A and B co-encapsulated at a 1:5 ratio in a drug delivery vehicle, then the required dosage of the preparation may be used directly for administration to the patient.

In another aspect, the invention is directed to a method to deliver a patient-specific synergistic or additive ratio of two or more therapeutic agents to said patient by administering the compositions of the invention.

In an additional aspect, the invention provides a kit comprising at least two pharmaceutical preparations; the first preparation containing at least a first therapeutic agent stably associated with a delivery vehicle and the second preparation containing at least a second therapeutic agent stably associated with a delivery vehicle, wherein the delivery vehicles have matched pharmacokinetics so as to maintain the initial ratio wherein the first and second pharmaceutical preparations can be combined at a non-antagonistic patient-specific ratio in order to treat said patient for a diseased condition.

### BRIEF DESCRIPTION OF THE DRAWINGS

FIG. 1 is a schematic view outlining the method of the invention for determining an appropriate ratio of therapeutic agents as tested on cultured cells.

FIG. 2 (A-E) illustrates graphs of A): a plot of combination index (CI) against fractions affected in A549 cells; B) and C): plots of combination index (CI) against irinotecan:carboplatin mole ratios; D) a bar graph of drug concentrations required to kill 90% of A459 cells by single drug administration or a combination of irinotecan and carboplatin; and E) a plot of irinotecan concentrations versus carboplatin concentrations.

FIG. 3A is a graph of combination index (CI) for irinotecan:5-FU at mole ratios of 1:10 (filled squares) and 1:1 (filled circles) as a function of the fraction of cultured HT29 cells affected (fₐ). FIG. 3B is a graph of CI for etoposide:carboplatin at mole ratios of 1:10 (filled diamonds) and 10:1 (filled squares) as a function of the fraction of cultured MCF-7 cells affected (fₐ).

FIG. 4 is a schematic of the methods used in the practice of this invention.

### MODES OF CARRYING OUT THE INVENTION¹

The disclosed invention involves determining a non-antagonistic ratio of therapeutic agents used in combination therapy and delivering that combination using coordinated pharmacokinetics. The ratio of therapeutic agents is thus synergistic or additive. The combination of therapeutic agents is selected by testing various therapeutic agents in various combinations and at various concentrations against cultured cells. Cultured cells used in the testing of the therapeutic agents are selected basis on a close molecular match to cells of a diseased patient. An individualized pharmaceutical composition is generated by matching test cells used to screen therapeutic agents to the molecular phenotype of a patient's diseased cells. Once selected, the therapeutic agents are formulated at the desired ratio in a manner that assures delivery of this ratio to target tissue in the individual patient.
¹ Abbreviations MTT: 3-(4,5-dimethylthiazol-2-yl)-2,5-diphenyl-2H tetrazolium bromide; CI: combination index; fₐ: fraction affected.

Determining the molecular phenotype of diseased cells starts with obtaining a biopsy sample or blood from the patient. The sample is then subjected to a battery of tests that identify the molecular phenotype (or genomic fingerprint) of the cells in the biopsy or blood. Once identified, the molecular phenotype of the patient sample is compared to the phenotype of a variety of cultured cell lines and at least one cultured cell line is selected based on its similarity to the patient sample. The cultured cell line is screened in vitro at various ratios of the therapeutic agents to determine an appropriate non-antagonistic ratio of agents to be used for that cell line and thus for that individual patient. The ratio is formulated into a composition of delivery vehicles such that the delivery vehicles have compatible pharmacokinetics to ensure maintenance of the ratio initially administered.

In a preferred embodiment, a single site is organized to assess and formulate therapeutic agent combinations as well to carry out techniques used for molecular phenotyping both the patient's and cultured cells. Therapeutic agent combinations or pharmaceutical preparations are preferably prepared from pre-formulated individual therapeutic agents in delivery vehicles with coordinated pharmacokinetics. Thus, in one illustrative embodiment, the site receives cells from an individual patient and identifies the molecular phenotype of those cells. The phenotype of the patient's cells is matched to the molecular phenotype of cultured cells. Cultured cells presenting a similar molecular phenotype to the patient's cells are assayed with various proposed therapeutic agent combinations at various ratios according to the methods described below. Using these methods the most favorable ratio of therapeutic agents effective against the cultured cells is identified. Ratio testing against the cultured cell lines can be performed before or after a patient's tissue or cell sample has been obtained.

After a preferred combination of therapeutic agents is identified and a preferred ratio of agents is determined, a pharmaceutical preparation containing the therapeutic agents and the delivery vehicle or vehicles is generated. Alternatively, individually formulated therapeutic agents along with information as to the ratio to be provided the patient may be provided to a care giver for later use.

The method of the invention typically involves identifying a patient-specific ratio of therapeutic agents in vitro which is non-antagonistic to cultured cells that have a molecular phenotype similar or identical to that of the patient. Ultimately this non-antagonistic ratio is supplied to the patient in a manner that will ensure that the ratio is maintained at the site of desired activity. The synergistic or additive ratio of therapeutic agents is determined by applying standard analytical tools to the results obtained when at least one ratio of two or more therapeutic agents is tested in vitro over a range of concentrations against the cultured cells. By way of illustration, individual therapeutic agents and various combinations thereof are tested for their biological effect on cultured cells, for example causing cell death or inhibiting cell growth, at various concentration levels.

In preferred embodiments, the concentration levels of therapeutic agents comprising a patient-specific ratio are plotted against the percentage cell survival to obtain a “combination index” (CI). The mathematics used are such that a CI of 1 (for example, a range from about 0.9-1.1) describes an additive effect of the reactive agents on a cultured cell line. A CI>1 (for example, a value that is approximately greater than 1.1) represents an antagonist effect of the reactive agents on a cultured cell line. A CI of <1 (i.e., for example, a value that is approximately less than 0.9) represents a synergistic effect of the reactive agents on a cultured cell line. (See Examples 1 and 2). The in vitro determination of non-antagonistic ratios for a combination of two or more therapeutic agents can be determined using the techniques discussed above.

The patient-specific ratio of therapeutic agents obtained in this way is provided to the patient by the pharmaceutical composition by encapsulating the agents in a pre-determined ratio in liposomes or other particulate forms. Delivery vehicles are selected that assure maintenance of the non-antagonistic ratio. The pharmaceutical preparations thus contain delivery vehicles that are preferably particulate in form and contain a desired ratio of therapeutic agents identified for an individual patient.

Therapeutic agents can be co-encapsulated so that they are contained in the same delivery vehicle. Alternatively, the therapeutic agents can be encapsulated in separate delivery vehicles. Delivery vehicles provided herein are such that consistent and coordinated delivery of the therapeutic components at the desired ratio is accomplished. Thus, the delivery of the therapeutic agents at the patient specific ratio is maintained using delivery vehicles that control the pharmacokinetics of therapeutic agent delivery such that the therapeutic agents are delivered to the patient at a pre-selected non-antagonistic ratio.

The term “encapsulation” refers to a stable association of therapeutic agent and the delivery vehicle. It is not necessary for the vehicle to surround the therapeutic agent or agents. Therapeutic agents need only be stably associated with the delivery vehicle when administered in vivo. And be released to achieve the predetermined patient specific therapeutic agent ratio. Thus, “stably associated with” and “encapsulated in” or “encapsulated with” or “co-encapsulated in or with” are intended to be synonymous terms. Accordingly, these terms are used interchangeably in this specification.

Suitable delivery vehicles include lipid carriers, liposomes, lipid micelles, lipoprotein micelles, lipid-stabilized emulsions, cyclodextrins, polymer nanoparticles, polymer microparticles, block copolymer micelles, polymer-lipid hybrid systems, derivatized single chain polymers, and the like. The delivery vehicles are selected for the ability to produce the release of the one or more therapeutic agents with the desired pharmacokinetics of each therapeutic agent being administered and to deliver the desired ratio of therapeutic agents to a target site.

The disclosed invention further involves generating a patient-specific pharmaceutical preparation containing a desired ratio of therapeutic agents that provides a non-antagonistic effect in vitro to selected cultured cells. Suitable cultured cells used to determine patient specific ratios for therapeutic agents are selected by determining the degree of molecular phenotype similarity between the patient's diseased cells and the cultured cells. The pharmaceutical preparation may be prepared by the same person or persons performing the molecular phenotyping and/or in vitro drug screening or different. The pharmaceutical preparation may be prepared by combining appropriate amounts of delivery vehicle-encapsulated therapeutic agents (either pre-formulated or formulated after in vitro analysis of cells) at the desired ratio or preparing or selecting delivery vehicles co-encapsulating the desired therapeutic agents at the desired ratio.

In Vitro Determination of Patient-Specific Non-Antagonistic Ratios

To prepare the pharmaceutical preparations described herein, the desired ratio of therapeutic agents contained in the delivery vehicles is determined for an individual patient. This determination is typically carried out in two steps. One step relates to determining the molecular phenotype of the patient. The molecular phenotype of a patient's diseased cells is matched with the molecular phenotype of at least one type of cultured cells. The other step of the process relates to screening the cultured cells against a number of combinations of therapeutic agents at multiple ratios. This screening program serves to identify a ratio of therapeutic agents that exhibits an optimal biological effect against the cultured cells. Desirably, the delivery ratio of therapeutic agents is synergistic or additive. Such ratios can be determined in vitro in the cultured cells using various mathematical models.

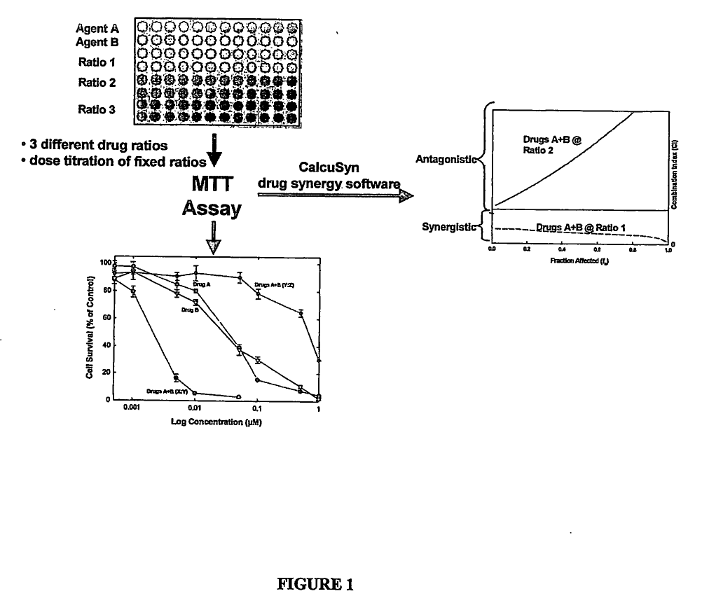

One general approach for screening cells in vitro to identify non-antagonistic ratios of therapeutic agents is shown in FIG. 1. As shown, therapeutic agents A and B are tested individually and together at two different ratios for their ability to cause cell death or cell stasis. In these experiments cell death or stasis was assessed using the MTT (3-[4,5-dimethylthiazol-2-yl]-2,5-diphenyltetrazolium) assay described below. Briefly, the dye MTT is reduced by living cells, producing a measurable change in optical density that is correlated to cell viability.

Correlations between the concentration of therapeutic agents A and B, and the two different combination ratios (Y:Z and X:Y) are plotted against cytotoxicity. Cytotoxicity is calculated as a percentage of surviving cells based on the survival of untreated control cells. A dose-dependent effect on cell survival both for the individual therapeutic agents and for the combinations was observed, as expected. Once this correlation has been established, the cell survival or fraction affected (fₐ) is used as a surrogate for concentration in calculating the CI.

The results of a CI calculation are also shown in FIG. 1. This CI index is calculated as a function of the fraction of cells affected according to the procedure of Chou and Talalay (Advance Enz. Regul. (1985) 22:27-55). In this hypothetical situation, the first ratio (X:Y) of therapeutic agents A plus B was non-antagonistic at all concentrations. In contrast, the combination of the therapeutic agents in the second ratio (Y:Z) was antagonistic. Thus, it is possible to provide a ratio of therapeutic agents A plus B (ratio 1) that is non-antagonistic regardless of concentration over a wide range. It is this ratio that is desirable to include in the compositions of the invention.

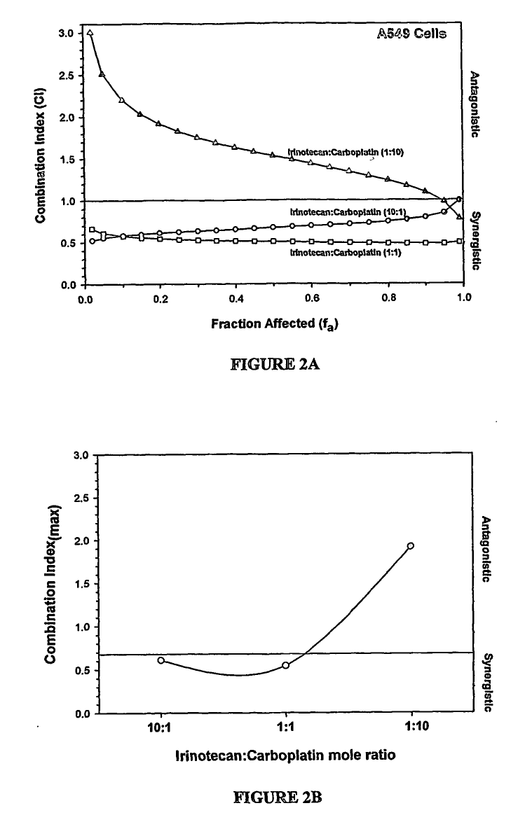

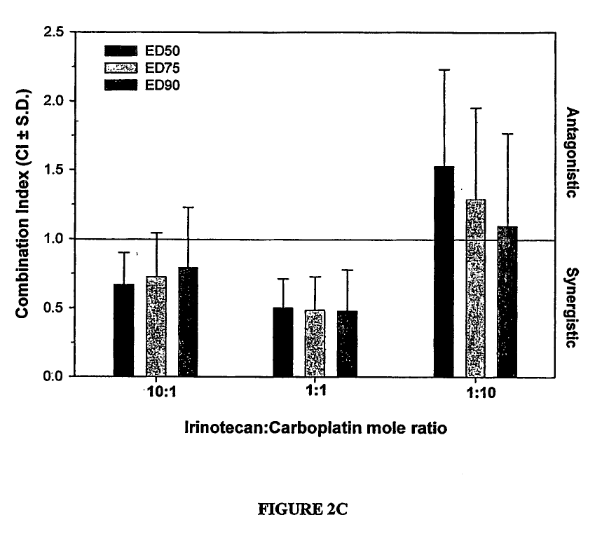

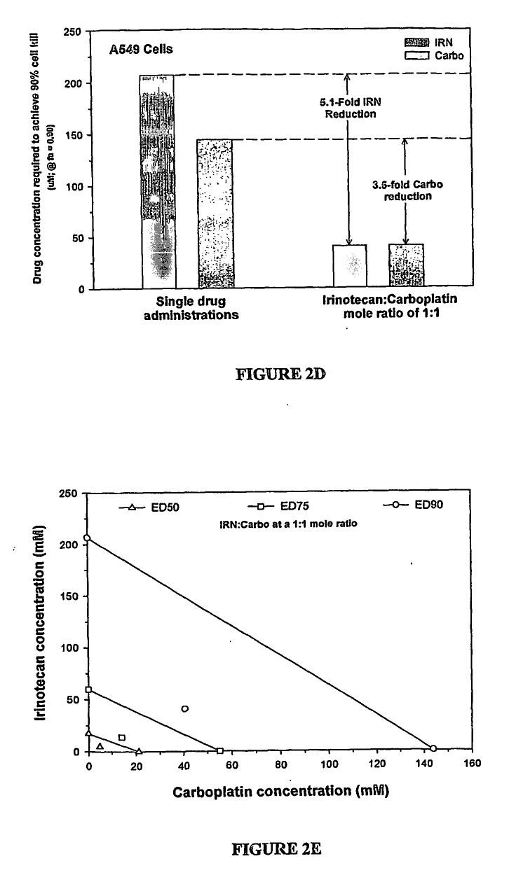

An alternative illustration of the effect of ratio and concentration on synergy by calculating a “CI maximum” for various ratios of therapeutic agents is discussed below. The “CI maximum” is defined as the CI value taken for a single fₐ value (between 0.2 and 0.8) where the greatest difference in CI values for the therapeutic agents at different ratios was observed. This is illustrated in FIGS. 2A and 2B. As shown, when the irinotecan/carboplatin ratio is 1:10, the CI differs most from that of the remaining ratios where the fraction affected value is 0.2. The CI value for this ratio at fₐ 0.2 is, as shown, approximately 2.0.

FIGS. 1 and 2 illustrate the in vitro determination of non-antagonistic ratios for a combination of only two therapeutic agents. Application of the same techniques described herein to combinations of three or more therapeutic agents provides a CI value over the concentration range in a similar manner. A two-therapeutic agent combination can be further used as a single pharmaceutical unit to determine synergistic or additive interactions with a third agent. In addition, a three-agent combination may be used as a unit to determine non-antagonistic interactions with a fourth agent, and so on.

Determination of ratios of therapeutic agents that display synergistic or additive effects may be carried out using various algorithms, based on the types of experimental data described below. These methods include isobologram methods (Loewe, et al., Arzneim-Forsch (1953) 3:285-290; Steel, et al., Int. J. Radiol. Oncol Biol. Phys. (1979) 5:27-55), the fractional product method (Webb, Enzyme and Metabolic Inhibitors (1963) Vol. 1, pp. 1-5. New York: Academic Press), the Monte Carlo simulation method, CombiTool, ComboStat and the Chou-Talalay median-effect method based on an equation described in Chou, J. Theor. Biol. (1976) 39:253-76; and Chou, Mol. Pharmacol. (1974) 10:235-247). Alternatives include surviving fraction (Zoli, et al., Int. J. Cancer (1999) 80:413-416), percentage response to granulocyte/macrophage-colony forming unit compared with controls (Pannacciulli, et al., Anticancer Res. (1999) 19:409-412) and others (Berenbaum, Pharmacol. Rev. (1989) 41:93-141; Greco, et al., Pharmacol Rev. (1995) 47:331-385).

The Chou-Talalay median-effect method is preferred. The analysis utilizes an equation wherein the dose that causes a particular effect, fₐ, is given by:
D=Dₘ[fₐ/(1−fₐ)]^{1/m}
in which D is the dose of the drug used, fₐ is the fraction of cells affected by that dose, Dₘ is the dose for median effect signifying the potency and m is a coefficient representing the shape of the dose-effect curve (m is 1 for first order reactions).

This equation can be further manipulated to calculate a combination index (CI) on the basis of the multiple drug effect equation as described by Chou and Talalay, Adv. Enzyme Reg. (1984) 22:27-55; and by Chou, et al., in: Synergism and Antagonism in Chemotherapy, Chou and Rideout, eds., Academic Press: New York 1991:223-244. A computer program (CalcuSyn) for this calculation is found in Chou and Chou (“Dose-effect analysis with microcomputers: quantitation of ED50, LD50, synergism, antagonism, low-dose risk, receptor ligand binding and enzyme kinetics”: CalcuSyn Manual and Software; Cambridge: Biosoft 1987).

The combination index equation is based on the multiple drug-effect equation of Chou-Talalay derived from enzyme kinetic models. An equation determines only the additive effect rather than synergism and antagonism. However, according to the CalcuSyn program, synergism is defined as a more than expected additive effect, and antagonism as a less than expected additive effect. Chou and Talalay in 1983 proposed the designation of CI=1 as the additive effect, thus from the multiple drug effect equation of two drugs, we obtain:
CI=(D)₁/(Dₓ)₁+(D)₂/(Dₓ)₂ [Eq. 1]
for mutually exclusive drugs that have the same or similar modes of action, and it is further proposed that
CI=(D)₁/(Dₓ)₁+(D)₂/(Dₓ)₂+(D₁)(D₂)/(Dₓ)₁(Dₓ)₂ [Eq. 2]
for mutually nonexclusive drugs that have totally independent modes of action. CI<1, =1, and >1 indicates synergism, additive effect, and antagonism, respectively. Equation 1 or equation 2 dictates that drug 1, (D)₁, and drug 2, (D)₂, (in the numerators) in combination inhibit x % in the actual experiment. Thus, the experimentally observed x % inhibition may not be a round number but most frequently has a decimal fraction. (Dₓ)₁ and (Dₓ)₂ (in the denominators) of equations 1 and 2 are the doses of drug 1 and drug 2 alone, respectively, inhibiting x %. For simplicity, mutual exclusivity is usually, but not always, assumed when more than two drugs are involved in combinations (CalcuSyn Manual and Software; Cambridge: Biosoft 1987).

After determining the underlying experimental data in vitro using the cultured cells, preferably, the combination index (CI) is plotted as a function of the fraction of cells affected (fₐ) as shown in FIG. 1 that is a surrogate parameter for concentration range. Preferred combinations of therapeutic agents are those that display synergy or additivity over a substantial range of fₐ values. Combinations of agents are selected that display synergy over at least 5% of the concentration range wherein greater than 1% of the cells are affected, i.e., an fₐ range greater than 0.01. Preferably, a larger portion of overall concentration exhibits a favorable CI; for example, 5% of an fₐ range of 0.2-0.8. More preferably 10% of this range exhibits a favorable CI. Even more preferably, 20% of the fₐ range, preferably over 50% and most preferably over at least 70% of the fₐ range of 0.2 to 0.8 are utilized in the compositions. Combinations that display synergy over a substantial range of fₐ values may be re-evaluated at a variety of agent ratios to define the optimal ratio to enhance the strength of the non-antagonistic interaction and increase the fₐ range over which synergy is observed.

While it would be desirable to have synergy over the entire range of concentrations over which cells are affected, it has been observed that in many instances, the results are considerably more reliable in an fₐ range of 0.2-0.8. Thus, although the synergy exhibited by combinations of the invention is set forth to exist within the broad range of 0.01 or greater, it is preferable that the synergy be established in the fₐ range of 0.2-0.8.

In one preferred embodiment, the given effect (fₐ) refers to cell death or cell stasis after application of a cytotoxic agent to the cultured cells. Cell death or viability may be measured using any accepted cytotoxicity assay. A list of suitable assays includes the following methods.

| CYTOTOXICITY ASSAY | REFERENCE |
|---|---|
| MTT assay | Mosmann, J. Immunol. Methods |
| | (1983) 65(1-2):55-63. |
| Trypan blue dye exclusion | Bhuyan, et al., Experimental Cell |
| | Research (1976) 97:275-280. |
| Radioactive tritium (³H)—thymidine | Senik, et al., Int. J. Cancer |
| incorporation or DNA intercalating | (1975) 16(6):946-959. |
| assay | |
| Radioactive chromium-51 release | Brunner, et al., Immunology |
| assay | (1968) 14:181-196. |
| Glutamate pyruvate transaminase, | Mitchell, et al., J. of Tissue |
| creatine phosphokinase and lactate | Culture Methods (1980) |
| dehydrogenase enzyme leakage | 6(3&4):113-116. |
| Neutral red uptake | Borenfreund and Puerner, Toxicol. |
| | Lett. (1985) 39:119-124. |
| Alkaline phosphatase activity | Kyle, et al., J. Toxicol. Environ. |
| | Health (1983) 12:99-117. |
| Propidium iodide staining | Nieminen, et al., Toxicol. Appl. |
| | Pharmacol. (1992) 115:147-155. |
| Bis-carboxyethyl-carboxyfluorescein | Kolber, et al., J. Immunol. |
| (BCECF) retention | Methods (1988) 108:255-264. |
| Mitochondrial membrane potential | Johnson, et al., Proc. Natl. Acad. |
| | Sci. USA (1980) 77:990-994. |
| Clonogenic Assays | Puck, et al., J. of Experimental |
| | Medicine (1956) 103:273-283. |
| LIVE/DEAD Viability/Cytotoxicity | Morris, Biotechniques (1990) |
| assay | 8:296-308. |
| Sulforhodamine B (SRB) assays | Rubinstein, et al., J. Natl. Cancer |
| | Instit. (1990) 82:1113-1118. |

As set forth above, the in vitro synergy studies on “cultured cells” is used to identify a ratio of therapeutic agents to be used on an individual patient by matching the molecular phenotype of said patient's cells with the molecular phenotype of said cultured cells. The molecular phenotype of the cultured cells and/or patient's cells may be carried out using a number of techniques well known to those in the art. For example, a technique commonly employed is diagnostic immunohistochemistry using tissue microarrays in which the cells or tissue harvested from a patient are screened for their DNA, RNA and/or protein expression patterns in order to identify the presence or absence of known biological markers. These methods are reviewed in Liu, et al., Amer. J. of Path. (2002) 161(5):1557-1565; Wick and Mills, Amer. J. Surg. Path. (2001) 25(9):1208-1210 and Golub, et al., Science (1999) 286:531-536, which are hereby incorporated by reference in their entirety.

Preferred Agent Combinations

Various combinations of therapeutic agents, having been found to satisfy the criteria for non-antagonistic effects set forth above, are then provided in the form of formulations of drug delivery vehicles. A “therapeutic agent” is a compound that alone, or in combination with other compounds, has a desirable effect on a subject affected by an unwanted condition or disease.

Certain therapeutic agents are favored for use in combination when the target disease or condition is cancer. Non-limiting examples are:

“Signal transduction inhibitors” that interfere with or prevents signals that cause cancer cells to grow or divide;

“Cytotoxic agents”;

“Cell cycle inhibitors” or “cell cycle control inhibitors” that interfere with the progress of a cell through its normal cell cycle, the life span of a cell, from the mitosis that gives it origin to the events following mitosis that divides it into daughter cells;

“Checkpoint inhibitors” that interfere with the normal function of cell cycle checkpoints, e.g., the S/G2 checkpoint, G2/M checkpoint and G1/S checkpoint;

“Topoisomerase inhibitors,” such as camptothecins, that interfere with topoisomerase I or II activity, enzymes necessary for DNA replication and transcription;

“Receptor tyrosine kinase inhibitors” that interfere with the activity of growth factor receptors that possess tyrosine kinase activity;

“Apoptosis inducing agents” that promote programmed cell death;

“Antimetabolites,” such as Gemcitabine or Hydroxyurea, that closely resemble an essential metabolite and therefore interfere with physiological reactions involving it;

“Telomerase inhibitors” that interfere with the activity of a telomerase, an enzyme that extends telomere length and extends the lifetime of the cell and its replicative capacity;

“Cyclin-dependent kinase inhibitors” that interfere with cyclin-dependent kinases that control the major steps between different phases of the cell cycle through phosphorylation of cell proteins such as histones, cytoskeletal proteins, transcription factors, tumor suppresser genes and the like;

“DNA damaging agents”;

“DNA repair inhibitors”;

“Anti-angiogenic agents” that interfere with the generation of new blood vessels or growth of existing blood vessels that occurs during tumor growth; and

“Mitochondrial poisons” that directly or indirectly disrupt mitochondrial respiratory chain function;

“Radionucleotides” that emit radiation,

“Radiosensitizers”; and

“Drug resistance modulators” that are either active or inactive on their own and sensitize a drug resistant target to the effects of one or more therapeutic agents.

Especially preferred combinations for treatment of tumors are the clinically approved combinations set forth hereinabove. As these combinations have already been approved for use in humans, reformulation to assure appropriate delivery is especially important.

Preferred agents that may be used in combination include DNA damaging agents such as carboplatin, cisplatin, cyclophosphamide, doxorubicin, daunorubicin, epirubicin, mitomycin C, mitoxantrone; DNA repair inhibitors including 5-fluorouracil (5-FU) or FUDR, gemcitabine and methotrexate; topoisomerase I inhibitors such as camptothecin, irinotecan and topotecan; S/G2 or G2/M checkpoint inhibitors such as bleomycin, docetaxel, doxorubicin, etoposide, paclitaxel, vinblastine, vincristine, vindesine and vinorelbine; G1/early-S checkpoint inhibitors; G2/M checkpoint inhibitors; receptor tyrosine kinase inhibitors such as genistein, trastuzumab, ZD1839; cytotoxic agents; apoptosis-inducing agents, cell cycle control inhibitors, and drug resistance modulators, such as the calcium channel blocker, verapamil and the immunosupressant, cyclosporin A.

A variety of antiviral chemotherapeutic agents can also be used with the present disclosure. For example, combinations of cytokines, acyclovir, amantidine, ribavirin, zidovudine, protease inhibitors, and others can be formulated into non-antagonistic combinations to prepare pharmaceutical preparations according to the present disclosure. Similarly, combinations of antibiotic compounds can also be formulated into pharmaceutical preparations according to the present disclosure.

The mechanism of action of one or more of the agents need not be known or may be incorrectly identified and still function using the methods disclosed herein. All patient-specific synergistic or additive combinations of agents are within the scope of the present invention. Preferably, for the treatment of a neoplasm, combinations that inhibit more than one mechanism that leads to uncontrolled cell proliferation are tested for their patient-specific use in accordance with this invention.

Particularly preferred combinations to be tested on cultured cells that can be matched to the molecular phenotype of cells from the diseased tissue or blood of a patient are DNA damaging agents in combination with DNA repair inhibitors, DNA damaging agents in combination with topoisomerase I or topoisomerase II inhibitors, topoisomerase I inhibitors in combination with S/G2 or G2/M checkpoint inhibitors, G1/S checkpoint inhibitors or CDK inhibitors in combination with G2/M checkpoint inhibitors, receptor tyrosine kinase inhibitors in combination with cytotoxic agents, apoptosis-inducing agents in combination with cytotoxic agents, apoptosis-inducing agents in combination with cell-cycle control inhibitors, G1/S or G2/M checkpoint inhibitors in combination with cytotoxic agents, topoisomerase I or II inhibitors in combination with DNA repair inhibitors, topoisomerase I or II inhibitors or telomerase inhibitors in combination with cell cycle control inhibitors, topoisomerase I inhibitors in combination with topoisomerase II inhibitors, and two cytotoxic agents in combination.

Preferred candidate combinations in general include those set forth hereinabove as already shown to be efficacious in the clinic as recognized by the FDA and those further suggested based on literature reports. While the candidate agents for use in the method of the invention are not limited to these specific combinations, those set forth hereinabove have been disclosed as suitable combination therapies, and are thus preferred for use in the methods and compositions of the present invention.

Some lipids are “therapeutic lipids” that are able to exert therapeutic effects such as inducing apoptosis. Included in this definition are lipids such as ether lipids, phosphatidic acid, phosphonates, ceramide and ceramide analogues, dihydroxyceramide, phytoceramide, sphingosine, sphingosine analogues, sphingomyelin, serine-containing lipids and sphinganine The term “serine-containing phospholipid” or “serine-containing lipid” as defined herein is a phospholipid in which the polar head group comprises a phosphate group covalently joined at one end to a serine and at the other end to a three-carbon backbone connected to a hydrophobic portion through an ether, ester or amide linkage. Included in this class are the phospholipids such as phosphatidylserine (PS) that have two hydrocarbon chains in the hydrophobic portion that are between 5-23 carbon atoms in length and have varying degrees of saturation. The term hydrophobic portion with reference to a serine-containing phospholipid or serine-containing lipid refers to apolar groups such as long saturated or unsaturated aliphatic hydrocarbon chains, optionally substituted by one or more aromatic, alicyclic or heterocyclic group(s). Combinations of therapeutic lipids and other agents can also be used to achieve synergistic or additive effects. Non-antagonistic combinations of agents may also be identified for their activity against microbial or viral infections as well as inflammatory disorders using similar techniques as described above.

Preparation of Delivery Vehicles Containing Encapsulated Drugs

When the appropriate ratios of therapeutic agents have been determined using the above methods, the therapeutic agents at the appropriate ratio are placed into a delivery vehicle composition wherein one or more delivery vehicles encapsulates one or more therapeutic agents. The delivery vehicles used in the pharmaceutical preparations need not be identical, but should produce coordinated pharmacokinetics for the encapsulated therapeutic agents. The delivery vehicles used in the pharmaceutical preparations are typically particles of sizes that, depending on their route of administration, can be suspended in an aqueous or other solvent. Such vehicles include, for example, lipid carriers, liposomes, cyclodextrins, polymer nanoparticles and polymer microparticles, including nanocapsules and nanospheres, block copolymer micelles, lipid stabilized emulsions, derivatized single-chain polymers, polymer-lipid hybrid systems, lipid micelles, lipoprotein micelles and the like.

As set forth above, the therapeutic agents are stably associated with the delivery vehicles in a patient specific pharmaceutical preparation. The therapeutic agents may be encapsulated together or stably associated with the same delivery vehicles or may be associated with different delivery vehicles in the same pharmaceutical preparation.

Liposomes can be prepared as described in Liposomes: Rational Design (A. S. Janoff ed., Marcel Dekker, Inc., N.Y.), or by additional techniques known to those knowledgeable in the art. Liposomes for use in this invention may be prepared to be of “low-cholesterol” (less than 30 mol %). Liposomes of the invention may also contain therapeutic lipids, which include ether lipids, phosphatidic acid, phosphonates, ceramide and ceramide analogues, sphingosine and sphingosine analogues and serine-containing lipids. Liposomes may also be prepared with surface stabilizing hydrophilic polymer-lipid conjugates such as polyethylene glycol-DSPE, to enhance circulation longevity. The incorporation of negatively charged lipids such as phosphatidylglycerol (PG) and phosphatidylinositol (PI) may also be added to liposome formulations to increase the circulation longevity of the carrier. These lipids may be employed to replace hydrophilic polymer-lipid conjugates as surface stabilizing agents. Embodiments of this invention may make use of cholesterol-free liposomes containing PG or PI to prevent aggregation thereby increasing the blood residence time of the carrier.

Micelles are self-assembling particles composed of amphipathic lipids or polymeric components that are utilized for the delivery of sparingly soluble agents present in the hydrophobic core. Various means for the preparation of micellar delivery vehicles are available and may be carried out with ease by one skilled in the art. For instance, lipid micelles may be prepared as described in Perkins, et al., Int. J. Pharm. (2000) 200(1):27-39 (incorporated herein by reference). Lipoprotein micelles can be prepared from natural or artificial lipoproteins including low and high-density lipoproteins and chylomicrons. Lipid-stabilized emulsions are micelles prepared such that they comprise an oil filled core stabilized by an emulsifying component such as a monolayer or bilayer of lipids. The core may comprise fatty acid esters such as triacylglycerol (corn oil). The monolayer or bilayer may comprise a hydrophilic polymer lipid conjugate such as DSPE-PEG. These delivery vehicles may be prepared by homogenization of the oil in the presence of the polymer lipid conjugate. Therapeutic agents that are incorporated into lipid-stabilized emulsions are generally poorly water-soluble. Synthetic polymer analogues that display properties similar to lipoproteins such as micelles of stearic acid esters or poly(ethylene oxide) block-poly(hydroxyethyl-L-aspartamide) and poly(ethylene oxide)-block-poly(hydroxyhexyl-L-aspartamide) may also be used in the practice of this invention (Lavasanifar, et al., J. Biomed. Mater. Res. (2000) 52:831-835).

Cyclodextrins comprise cavity-forming, water-soluble, oligosaccharides that can accommodate water-insoluble drugs in their cavities. Therapeutic agents can be encapsulated into cyclodextrins using procedures known to those skilled in the art. For example, see Atwood, et al., Eds., “Inclusion Compounds,” Vols. 2 & 3, Academic Press, NY (1984); Bender, et al., “Cyclodextrin Chemistry,” Springer-Verlag, Berlin (1978); Szeitli, et al., “Cyclodextrins and Their Inclusion Complexes,” Akademiai Kiado, Budapest, Hungary (1982) and WO 00/40962.

Nanoparticles and microparticles may comprise a concentrated core of drug that is surrounded by a polymeric shell (nanocapsules) or as a solid or a liquid dispersed throughout a polymer matrix (nanospheres). General methods of preparing nanoparticles and microparticles are described by Soppimath, et al. (J. Control Release (2001) 70(1-2):1-20), each of which is hereby incorporated by reference in their entirety. Other polymeric delivery vehicles that may be used include block copolymer micelles that comprise a drug containing a hydrophobic core surrounded by a hydrophilic shell; they are generally utilized as carriers for hydrophobic drugs and can be prepared as found in Allen, et al., Colloids and Surfaces B: Biointerfaces (1999) November 16(1-4):3-27. Polymer-lipid hybrid systems consist of a polymer nanoparticle surrounded by a lipid monolayer. The polymer particle serves as a cargo space for the incorporation of hydrophobic drugs while the lipid monolayer provides a stabilizing interference between the hydrophobic core and the external aqueous environment. Polymers such as polycaprolactone and poly(d,1-lactide) may be used while the lipid monolayer is typically composed of a mixture of lipid. Suitable methods of preparation are similar to those referenced above for polymer nanoparticles. Derivatized single chain polymers are polymers adapted for covalent linkage of a biologically active agent to form a polymer-drug conjugate. Numerous polymers have been proposed for synthesis of polymer-drug conjugates including polyaminoacids, polysaccharides such as dextrin or dextran, and synthetic polymers such as N-(2-hydroxypropyl)methacrylamide (HPMA) copolymer. Suitable methods of preparation are detailed in Veronese and Morpurgo, IL Farmaco (1999) 54(8):497-516 and are incorporated by reference herein.

For intravenous administration, delivery vehicles are typically about 4-6,000 nm in diameter. Preferred diameters are about 5-500 nm in diameter, more preferably 5-200 nm in diameter. For inhalation, intra-thecal, intra-articular, intra-arterial, intra-peritoneal or subcutaneous administration, delivery vehicles are typically from 4 μm to an excess of 50 μm. Delivery vehicle compositions designed for intra-ocular administration are generally smaller.

The therapeutic agents are “encapsulated” in the delivery vehicles. “Encapsulation,” includes covalent or non-covalent association of an agent with the delivery vehicle. This can be by interaction of the agent with the outer layer or layers of the delivery vehicle or entrapment of an agent within the delivery vehicle, equilibrium being achieved between different portions of the delivery vehicle. For example, for liposomes, encapsulation of an agent can be by association of the agent by interaction with the bilayer of the liposomes through covalent or non-covalent interaction with the lipid components or entrapment in the aqueous interior of the liposome, or in equilibrium between the internal aqueous phase and the bilayer. For polymer-based delivery vehicles, encapsulation can refer to covalent linkage of an agent to a linear or non-linear polymer. Further, non-limiting examples include the dispersion of agent throughout a polymer matrix, or the concentration of drug in the core of a nanocapsule, a block copolymer micelle or a polymer-lipid hybrid system. “Loading” refers to the act of encapsulating one or more agents into a delivery vehicle.

Encapsulation of the desired combination can be achieved either through encapsulation in separate delivery vehicles or within the same delivery vehicle. Preferably, encapsulation into delivery vehicles is done prior to screening a patient for their optimal non-antagonistic ratio. Where encapsulation into separate delivery vehicles, such as liposomes, is desired, the lipid composition of each liposome may be quite different to allow for similar pharmacokinetics of each drug. By altering the vehicle composition, release rates of encapsulated drugs can be matched to allow non-antagonistic ratios of the drugs to be delivered to the tumor site. Methods for altering release rates include increasing the acyl-chain length of vesicle forming lipids to improve drug retention, controlling the exchange of surface grafted hydrophilic polymers such as PEG out of the liposome membrane and incorporating membrane-rigidifying agents such as sterols or sphingomyelin into the membrane. It should be apparent to those skilled in the art that if a first and second drug are desired to be administered at a specific drug ratio and if the second drug is retained poorly within the liposome composition of the first drug (e.g., DMPC/Chol), that improved pharmacokinetics may be achieved by encapsulating the second drug in a liposome composition with lipids of increased acyl chain length (e.g., DSPC/Chol). Alternatively, two or more agents may be encapsulated within the same delivery vehicle.

Techniques for encapsulation are dependent on the nature of the delivery vehicles. For example, therapeutic agents may be loaded into liposomes using both passive and active loading methods.

Passive methods of encapsulating agents in liposomes involve encapsulating the agent during the preparation of the liposomes. In this method, the drug may be membrane associated or encapsulated within an entrapped aqueous space. This includes a passive entrapment method described by Bangham, et al., J. Mol. Biol. (1965) 12:238, where the aqueous phase containing the agent of interest is put into contact with a film of dried vesicle-forming lipids deposited on the walls of a reaction vessel. Upon agitation by mechanical means, swelling of the lipids will occur and multilamellar vesicles (MLV) will form. Using extrusion, the MLV's can be converted to large unilamellar vesicles (LUV) or small unilamellar vesicles (SUV). Another method of passive loading that may be used includes that described by Deamer and Bangham, Biochim. Biophys. Acta (1976) 443:629. This method involves dissolving vesicle-forming lipids in ether and, instead of first evaporating the ether to form a thin film on a surface, this film being thereafter put into contact with an aqueous phase to be encapsulated, the ether solution is directly injected into said aqueous phase and the ether is evaporated afterwards, whereby liposomes with encapsulated agents are obtained. A further method that may be employed is the Reverse Phase Evaporation (REV) method described by Szoka and Papahadjopoulos, P.N.A.S. (1978) 75:4194, in which a solution of lipids in a water insoluble organic solvent is emulsified in an aqueous carrier phase and the organic solvent is subsequently removed under reduced pressure.

Other methods of passive entrapment that may be used include subjecting liposomes to successive dehydration and rehydration treatment, or freezing and thawing. Dehydration is carried out by evaporation or freeze-drying. This technique is disclosed by Kirby, et al., Biotechnology (1984) 979-984. Also, Shew and Deamer (Biochim. et Biophys. Acta (1985) 816:1-8) describe a method wherein liposomes prepared by sonication are mixed in aqueous solution with the solute to be encapsulated, and the mixture is dried under nitrogen in a rotating flask. Upon rehydration, large liposomes are produced in which a significant fraction of the solute has been encapsulated.

Passive encapsulation of two or more agents is possible for many drug combinations. This approach is limited by the solubility of the drugs in aqueous buffer solutions and the large percentage of drug that is not trapped within the delivery system. The loading may be improved by co-lyophilizing the drugs with the lipid sample and rehydrating in the minimal volume allowed to solubilize the drugs. The solubility may be improved by varying the pH of the buffer, increasing temperature or addition or removal of salts from the buffer.

Active methods of encapsulating may also be used. For example, liposomes may be loaded according to a metal-complexation or pH gradient loading technique. With pH gradient loading, liposomes are formed which encapsulate an aqueous phase of a selected pH. Hydrated liposomes are placed in an aqueous environment of a different pH selected to remove or minimize a charge on the drug or other agent to be encapsulated. Once the drug moves inside the liposome, the pH of the interior results in a charged drug state, which prevents the drug from permeating the lipid bilayer, thereby entrapping the drug in the liposome.

To create a pH gradient, the original external medium can be replaced by a new external medium having a different concentration of protons. The replacement of the external medium can be accomplished by various techniques, such as, by passing the lipid vesicle preparation through a gel filtration column, e.g., a Sephadex G-50 column, which has been equilibrated with the new medium (as set forth in the examples below), or by centrifugation, dialysis, or related techniques. The internal medium may be either acidic or basic with respect to the external medium.

After establishment of a pH gradient, a pH gradient loadable agent is added to the mixture and encapsulation of the agent in the liposome occurs as described above.

Loading using a pH gradient may be carried out according to methods described in U.S. Pat. Nos. 5,616,341, 5,736,155 and 5,785,987 incorporated herein by reference. A preferred method of pH gradient loading is the citrate-based loading method utilizing citrate as the internal buffer at a pH of 2-6 and a neutral external buffer.

Various methods may be employed to establish and maintain a pH gradient across a liposome all of which are incorporated herein by reference. This may involve the use of ionophores that can insert into the liposome membrane and transport ions across membranes in exchange for protons (see for example U.S. Pat. No. 5,837,282). Compounds encapsulated in the interior of the liposome that are able to shuttle protons across the liposomal membrane and thus set up a pH gradient (see for example U.S. Pat. No. 5,837,282) may also be utilized. These compounds comprise an ionizable moiety that is neutral when deprotonated and charged when protonated. The neutral deprotonated form (which is in equilibrium with the protonated form) is able to cross the liposome membrane and thus leave a proton behind in the interior of the liposome and thereby cause an decrease in the pH of the interior. Examples of such compounds include methylammonium chloride, methylammonium sulfate, ethylenediammonium sulfate (see U.S. Pat. No. 5,785,987) and ammonium sulfate. Internal loading buffers that are able to establish a basic internal pH, can also be utilized. In this case, the neutral form is protonated such that protons are shuttled out of the liposome interior to establish a basic interior. An example of such a compound is calcium acetate (see U.S. Pat. No. 5,939,096).

Two or more agents may be loaded into a liposome using the same active loading methods or may involve the use of different active loading methods. For instance, metal complexation loading may be utilized to actively load multiple agents or may be coupled with another active loading technique, such as pH gradient loading. Metal-based active loading typically uses liposomes with passively encapsulated metal ions (with or without passively loaded therapeutic agents). Various salts of metal ions are used, presuming that the salt is pharmaceutically acceptable and soluble in an aqueous solutions. Actively loaded agents are selected based on being capable of forming a complex with a metal ion and thus being retained when so complexed within the liposome, yet capable of loading into a liposome when not complexed to metal ions. Agents that are capable of coordinating with a metal typically comprise coordination sites such as amines, carbonyl groups, ethers, ketones, acyl groups, acetylenes, olefins, thiols, hydroxyl or halide groups or other suitable groups capable of donating electrons to the metal ion thereby forming a complex with the metal ion. Examples of active agents which bind metals include, but are not limited to, quinolones such as fluoroquinolones; quinolones such as nalidixic acid; anthracyclines such as doxorubicin, daunorubicin and idarubicin; amino glycosides such as kanamycin; and other antibiotics such as bleomycin, mitomycin C and tetracycline; and nitrogen mustards such as cyclophosphamide, thiosemicarbazones, indomethacin and nitroprusside; camptothecins such as topotecan, irinotecan, lurtotecan, 9-aminocamptothecin, 9-nitrocamptothecin and 10-hydroxycamptothecin; and podophyllotoxins such as etoposide. Uptake of an agent may be established by incubation of the mixture at a suitable temperature after addition of the agent to the external medium. Depending on the composition of the liposome, temperature and pH of the internal medium, and chemical nature of the agent, uptake of the agent may occur over a time period of minutes or hours. Methods of determining whether coordination occurs between an agent and a metal within a liposome include spectrophotometric analysis and other conventional techniques well known to those of skill in the art.

Furthermore, liposome loading efficiency and retention properties using metal-based procedures carried out in the absence of an ionophore in the liposome are dependent on the metal employed and the lipid composition of the liposome. By selecting lipid composition and a metal, loading or retention properties can be tailored to achieve a desired loading or release of a selected agent from a liposome.

Passive and active loading methods may be combined sequentially in order to load multiple drugs into a delivery vehicle. By way of example, liposomes containing a passively entrapped platinum drug such as cisplatin in the presence of MnCl₂ may subsequently be used to actively encapsulate an anthracycline such as doxorubicin into the interior of the liposome. This method is likely to be applicable to numerous drugs that are encapsulated in liposomes through passive encapsulation.

Preparing Patient-Specific Pharmaceutical Preparations

Once a patient-specific ratio of at least two therapeutic agents has been identified, there are a number of ways in which a pharmaceutical preparation designed for administration to said patient can be prepared. First, the therapeutic agents may have been pre-formulated in delivery vehicles prior to the in vitro identification of a patient-specific ratio using the methods above, or alternatively, they may be encapsulated in delivery vehicles after in vitro analysis. The therapeutic agents may be co-encapsulated in the same delivery vehicle or separately encapsulated. If the therapeutic agents are encapsulated in separate delivery vehicles, the composition of the delivery vehicles must allow for similar pharmacokinetics of each therapeutic agent to be administered. A person authorized to prepare said pharmaceutical preparation, such as but not limited to, a laboratory technician, pharmacist, nurse, physician and the like may prepare the patient-specific pharmaceutical preparation at the site of in vitro screening and molecular phenotyping or at a distant site, including a pharmacy or hospital.

The preparation can be prepared by combining appropriate amounts of separately-encapsulated therapeutic agents (pre-formulated or formulated after in vitro screening of cultured cells and molecular phenotyping of cultured and patients' cells) at the desired patient-specific ratio and placing them in a vial labeled for said patient; or, if a delivery vehicle is prepared with the therapeutic agents co-encapsulated at the desired ratio (pre-formulated or formulated after in vitro screening of cultured cells and molecular phenotyping of cultured and patients' cells), the required dosage of this preparation may be placed in a vial and labeled for said patient.

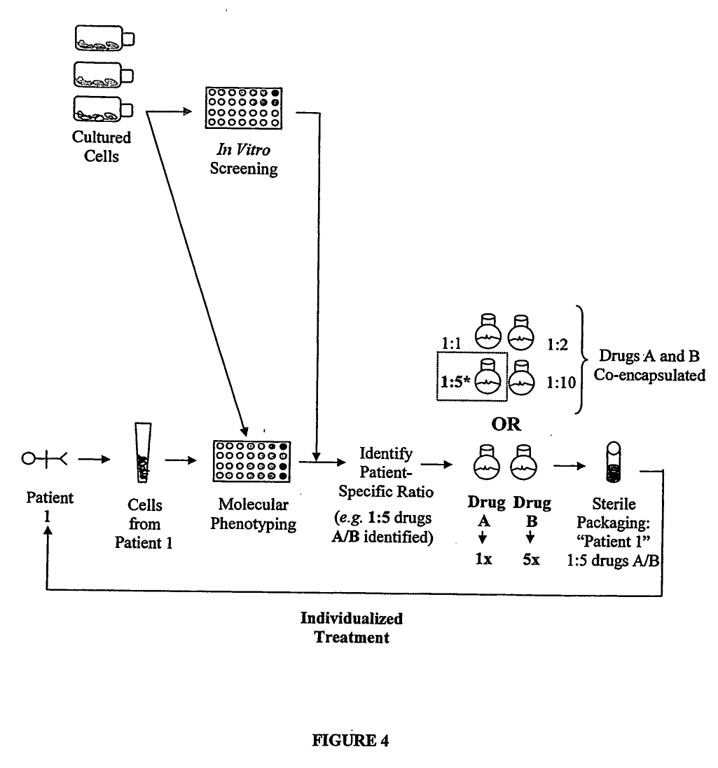

As outlined in FIG. 4, a patient-specific ratio was identified using in vitro screening of cultured cells that match the molecular/cellular phenotypes of an individual patient. In this illustration, the patient specific ratio was determined to be a 1:5 ratio of therapeutic agents A and B. The details of this ratio are transferred to a person qualified to make a pharmaceutical preparation for the patient in question. This person may either combine one part of therapeutic agent A (pre-formulated or post-in vitro-screening and/or post-molecular phenotyping-formulated in a delivery vehicle suitable for therapeutic agent A) with five parts of therapeutic agent B (pre-formulated or later formulated in a delivery vehicle suitable for therapeutic agent B) and place it in a vial labeled for said patient. Alternatively, if a delivery vehicle is already prepared with a 1:5 ratio of therapeutic agents A and B co-encapsulated (again, pre-formulated or formulated after molecular phenotyping and screening), the required dosage of this preparation may be placed in a vial and labeled for the patient. Preferably, pre-formulated delivery vehicle-encapsulated drugs will be utilized. The vials specific for each patient should be sterilized prior to administration of the patient-specific treatment to said patient. The individual drug preparations should also be sterilized and sterile techniques should be used throughout the practice of generating the patient-specific pharmaceutical preparation.

The “patient-specific pharmaceutical” preparation or composition therefore refers to a formulation containing a combination of at least two therapeutic agents, either co-encapsulated or separately encapsulated in delivery vehicles, that have been combined at a ratio optimized for its non-antagonistic effects on cultured cells that have a molecular phenotype similar or identical to the molecular phenotype of cells harvested from the diseased tissue or blood of said patient (i.e., “patient-specific ratio”). Once the encapsulated therapeutic agents have been combined at the patient-specific ratio to generate a patient-specific pharmaceutical preparation, the pharmaceutical preparation may be either directly administered to said patient, transferred to the site of said patient and/or stored for future administration to said patient. Furthermore, many combination therapies require multiple administrations of the same pharmaceutical preparation; therefore, the above pharmaceutical preparation may be duplicated (and stored, if necessary) for such cases.

Therapeutic Uses of Delivery Vehicle Compositions Encapsulating Multiple Agents

The ratio-specific delivery vehicle compositions described herein can be used to treat a variety of diseases in a variety of subjects. Suitable subjects for treatment according to the methods and compositions described herein include mammals generally, such as humans, livestock or domestic animals, domesticated avian subjects such as chickens and ducks, and laboratory animals for research use. Examples of medical uses of the compositions of the present invention include treating cancer, treating cardiovascular diseases such as hypertension, cardiac arrhythmia and restenosis, treating bacterial, viral, fungal or parasitic infections, treating and/or preventing diseases through the use of the compositions of the present inventions as vaccines, treating inflammation or treating autoimmune diseases.

In one embodiment, delivery vehicle compositions in accordance with this invention are preferably used to treat neoplasms. Delivery of formulated drug to a tumor site is achieved by administration of liposomes or other particulate delivery systems. Preferably liposomes have a diameter of less than 200 nm. Tumor vasculature is generally leakier than normal vasculature due to fenestrations or gaps in the endothelia. This allows the delivery vehicles of 200 nm or less in diameter to penetrate the discontinuous endothelial cell layer and underlying basement membrane surrounding the vessels supplying blood to a tumor. Selective accumulation of the delivery vehicles into tumor sites following extravasation leads to enhanced drug delivery and therapeutic effectiveness. Because carriers extravasate, it can be assumed that the carrier drug-to-drug ratio determined in the blood will be comparable to the carrier drug-to-drug ratio in the extravascular space.

Administering Compositions of the Invention In Vivo

As mentioned above, the ratio-specific delivery vehicle compositions of the present invention may be administered to warm-blooded animals, including humans as well as to domestic avian species. For treatment of human ailments, a qualified physician will determine how the patient-specific compositions of the present invention should be utilized with respect to dose, schedule and route of administration using established protocols. Such applications may also utilize dose escalation should agents encapsulated in delivery vehicle compositions of the present invention exhibit reduced toxicity to healthy tissues of the subject.

Preferably, the patient-specific pharmaceutical compositions of the present invention are administered parenterally, i.e., intraarterially, intravenously, intraperitoneally, subcutaneously, or intramuscularly. More preferably, the pharmaceutical compositions are administered intravenously or intraperitoneally by a bolus injection. For example, see Rahman, et al., U.S. Pat. No. 3,993,754; Sears, U.S. Pat. No. 4,145,410; Papahadjopoulos, et al., U.S. Pat. No. 4,235,871; Schneider, U.S. Pat. No. 4,224,179; Lenk, et al., U.S. Pat. No. 4,522,803; and Fountain, et al., U.S. Pat. No. 4,588,578, all of which are incorporated by reference in their entirety.

In other methods, the patient-specific pharmaceutical or cosmetic preparations of the present invention can be contacted with the target tissue by direct application of the preparation to the tissue. The application may be made by topical, “open” or “closed” procedures. By “topical”, it is meant the direct application of the multi-drug preparation to a tissue exposed to the environment, such as the skin, oropharynx, external auditory canal, and the like. “Open” procedures are those procedures that include incising the skin of a patient and directly visualizing the underlying tissue to which the pharmaceutical preparations are applied. This is generally accomplished by a surgical procedure, such as a thoracotomy to access the lungs, abdominal laparotomy to access abdominal viscera, or other direct surgical approach to the target tissue. “Closed” procedures are invasive procedures in which the internal target tissues are not directly visualized, but accessed via inserting instruments through small wounds in the skin. For example, the preparations may be administered to the peritoneum by needle lavage. Alternatively, the preparations may be administered through endoscopic devices.

Pharmaceutical compositions comprising delivery vehicles encapsulating patient-specific ratios of drugs as outlined in the present invention are prepared according to standard techniques and may comprise water, buffered water, 0.9% saline, 0.3% glycine, 5% dextrose and the like, including glycoproteins for enhanced stability, such as albumin, lipoprotein, globulin, and the like. These compositions may be sterilized by conventional, well-known sterilization techniques. The resulting aqueous solutions may be packaged for use or filtered under aseptic conditions and lyophilized, the lyophilized preparation being combined with a sterile aqueous solution prior to administration. The compositions may contain pharmaceutically acceptable auxiliary substances as required to approximate physiological conditions, such as pH adjusting and buffering agents, tonicity adjusting agents and the like, for example, sodium acetate, sodium lactate, sodium chloride, potassium chloride, calcium chloride, and the like. Additionally, the delivery vehicle suspension may include lipid-protective agents which protect lipids against free-radical and lipid-peroxidative damages on storage. Lipophilic free-radical quenchers, such as alpha-tocopherol and water-soluble iron-specific chelators, such as ferrioxamine, are suitable. Leucovorin may also be administered with compositions of the invention through standard techniques to enhance the life span of administered fluoropyrimidines.

The concentration of delivery vehicles in the patient-specific pharmaceutical formulations can vary widely, such as from less than about 0.05%, usually at or at least about 2-5% to as much as 10 to 30% by weight and will be selected primarily by fluid volumes, viscosities, and the like, in accordance with the particular mode of administration selected. For example, the concentration may be increased to lower the fluid load associated with treatment. Alternatively, delivery vehicles composed of irritating lipids may be diluted to low concentrations to lessen inflammation at the site of administration. For diagnosis, the amount of delivery vehicles administered will depend upon the particular label used, the disease state being diagnosed and the judgment of the clinician.

Preferably, the pharmaceutical compositions of the present invention are administered intravenously. Dosage for the delivery vehicle formulations will depend on the ratio of drug to lipid and the administrating physician's opinion based on age, weight, and condition of the patient.

In addition to pharmaceutical compositions, suitable formulations for veterinary use may be prepared and administered in a manner suitable to the subject. Preferred veterinary subjects include mammalian species, for example, non-human primates, dogs, cats, cattle, horses, sheep, and domesticated fowl. Subjects may also include laboratory animals, for example, in particular, rats, rabbits, mice, and guinea pigs.

The following examples are offered to illustrate but not to limit the invention. The preferred embodiments herein described are not intended to be exhaustive or to limit the scope of the invention to the precise forms disclosed. They are chosen and described to best explain the principles of the invention and its application and practical use to allow others skilled in the art to comprehend its teachings.

### EXAMPLES

### Example 1

### Multiple Representation of Dose-Effect Analysis

A critical part of this invention is identifying combinations of drugs that exhibit non-antagonistic effects to cultured cells, which have been shown either before or after drug screening to display a molecular phenotype similar or identical to the molecular phenotype of cells that have been harvested from diseased patients. To demonstrate the steps involved in the in vitro screening analysis of drug combinations on said cultured cells, a combination of irinotecan and carboplatin was tested at various ratios for their effects on cultured A549 cells. In a preferred embodiment of the invention, the method involves utilizing the following in vitro screening analysis on cultured cells, that exhibit a molecular phenotype similar or identical to the molecular phenotype of cells harvested from the diseased tissue or blood of an individual patient, to better identify particular drug combinations on a patient-by-patient basis.

Quantitative analysis of the relationship between an amount (dose or concentration) of drug and its biological effect as well as the joint effect of drug combinations can be measured and reported in a number of ways. FIG. 2 illustrates 5 such methods using, as an example, irinotecan and carboplatin. Based on Chou and Talalay's theory of dose-effect analysis, a “median-effect equation” has been used to calculate a number of biochemical equations that are extensively used in the art. Derivations of this equation have given rise to higher order equations such as those used to calculate Combination Index (CI). As mentioned previously, CI can be used to determine if combinations of more than one drug and various ratios of each combination are antagonistic, additive or synergistic. CI plots are typically illustrated with CI representing the y-axis versus the proportion of cells affected (cultured or patient cells), or fraction affected (fₐ), on the x-axis. FIG. 2A demonstrates that a 1:10 mole ratio of irinotecan/carboplatin is antagonistic (CI>1.1), while 1:1 and 10:1 have a synergistic effect (CI<0.9).

The present applicants have also designed an alternative method of representing the dependency of CI on the drug ratios used. The maximum CI value is plotted against each ratio to better illustrate trends in ratio-specific effects for a particular combination as seen in FIG. 2B. The CI maximum is the CI value taken at a single fₐ value (between 0.2 and 0.8) where the greatest difference in CI values for the drugs at different ratios was observed.

Because the concentrations of drugs used for an individual ratio play a role in determining the effect (i.e., synergism or antagonism), it can also be important to measure the CI at various concentrations. These concentrations, also referred to as “Effective Doses” (ED) by Chou-Talalay, are the concentration of drug required to affect a designated percent of the cells in an in vitro assay, i.e., ED₅₀ is the concentration of drug required to affect 50% of the cells relative to a control or untreated cell population. As shown in FIG. 2C, trends in concentration-effect are readily distinguished between the various ratios. The error bars shown represent one standard deviation around the mean and is determined directly through the CalcuSyn program.

A synergistic interaction between two or more drugs has the benefit that it can lower the amount of each drug required in order to result in a positive effect, otherwise known as “dose-reduction.” Chou and Talalay's “dose-reduction index” (DRI) is a measure of how much the dose of each drug in a synergistic combination may be reduced at a given effect level compared with the doses for each drug alone. DRI has been important in clinical situations, where dose-reduction leads to reduced toxicity for the host while maintaining therapeutic efficacy. The plot in FIG. 2D shows that the concentrations of irinotecan and carboplatin required to achieve a 90% cell kill on their own is significantly higher than their individual concentrations required when they are combined at a non-antagonistic ratio.

Furthermore, the aforementioned data can be represented in a classical isobologram (FIG. 2E). Isobolograms have the benefit that they can be generated at different ED values; however, they become more difficult to read as more effect levels are selected for interpretation. For this reason, data is preferably presented in accordance with the types of plots shown in FIGS. 2A and 2B.

### Example 2

### Therapeutic Effects are Dependent upon Concentrations

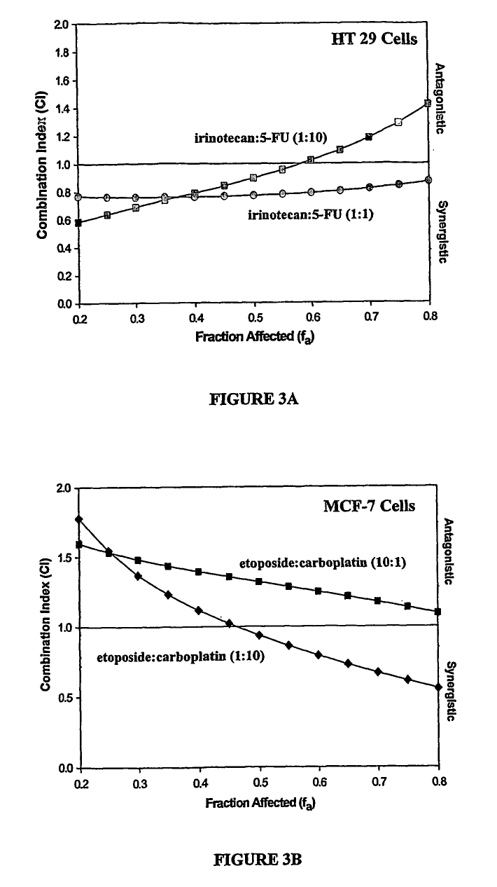

As mentioned previously, investigators of the present invention recognize that drug concentrations play a role in determining how a particular drug ratio may affect treated cells. To demonstrate this concept, drug combinations of irinotecan and 5-fluorouracil (5-FU) at mole ratios of 1:1 and 1:10 and etoposide and carboplatin at mole ratios of 10:1 and 1:10 were tested for additive, synergistic or antagonistic effects on cultured cells using the standard tetrazolium-based colorimetric MTT cytotoxicity assay and the median-effect analysis. In preferred embodiments of the invention, testing for non-antagonistic or antagonistic ratios of combinations of such therapeutic agents is performed on cultured cells, which have been shown either before or after drug screening to exhibit a molecular phenotype similar or identical to the molecular phenotype of cells that have been harvested from the diseased tissue or blood of a patient. For the purpose of illustration, cultured HT29 or MCF-7 cells were exposed to single agents as well as agents in combination at defined ratios to show the characteristics of drug ratios and concentrations. Eight drug concentrations were utilized for single agents and combinations. Optical density values were obtained from the MTT assay, converted into a percentage of the control, averaged and then converted into fraction affected values. Dose and fraction affected values were entered into CalcuSyn which yielded the CI versus fₐ graph, shown in FIG. 3.

FIG. 3A shows that irinotecan and 5-FU at a mole ratio of 1:1 was non-antagonistic (CI<0.9) over the entire range of concentrations as measured by the fraction-affected dose. In contrast at a mole ratio of 1:10, the same two drugs were non-antagonistic at low concentrations (low Fₐ values), yet antagonistic (CI>1.1) at higher concentrations (higher Fₐ values). As seen in FIG. 3B, etoposide and carboplatin were antagonistic at a mole ratio of 10:1 over the entire concentration range. In contrast, at a 1:10 mole ratio, etoposide and carboplatin were antagonistic at low concentrations while non-antagonistic at higher concentrations.

These results thus demonstrate that synergy is highly dependent on not only the ratio of the agents to one another but also their concentrations.

### Example 3

### Patient-Specific Generation and Administration of Therapeutic Ratios

The method of the invention further involves generating pharmaceutical preparations specific to an individual patient which is suitable for administration to said patient. Either before or after the diseased tissue or blood in a patient is removed and characterized by molecular phenotyping and matched against cultured cells with similar or identical molecular phenotypes, the cultured cells are screened as described in Examples 1 and 2 to identify an optimal non-antagonistic ratio of therapeutic agents. The optimal ratio identified for the cultured cells, which match the patient's molecular phenotype, is then chosen as the “patient-specific ratio” to be used for administration to said patient. Preparations for administration to patients can be performed in a number of ways. For example, in the case of two patients with similar cancers:

Patient one has a tumor biopsy that was analyzed in vitro as above, using molecular phenotyping and drug screening on cultured cells, to determine that a 1:5 ratio of drugs A-to-B should provide optimal non-antagonistic effects to patient one's cancerous cells (see FIG. 4). Patient two has a tumor biopsy that was also analyzed in vitro as above, using molecular phenotyping and drug screening on cultured cells, yet a 1:2 ratio of therapeutic agents A and B was identified as being an optimal non-antagonistic ratio for patient two's cancerous cells. Any person authorized to generate the pharmaceutical preparations to be administered to patient one may then combine one part of a formulation of therapeutic agent A (either pre-formulated or formulated after identification of the patient-specific ratio) with 5 parts of a formulation of therapeutic agent B (either pre-formulated or formulated after identification of the patient-specific ratio), provided that the therapeutic agent formulations are at doses and quality (i.e., sterile) approved for use in humans, and either administer the preparation to patient one (if authorized) or place the combined preparation in a vial labeled for patient one (as outlined in FIG. 4).

Alternatively, that person may place in a vial labeled for patient one, an appropriate amount of a delivery vehicle formulation that contains therapeutic agent A and B co-encapsulated at a 1:5 ratio (either pre-formulated or formulated after identification of patient one's specific ratio), providing that the amount and quality of the formulation placed in the vial is approved for use in humans. The same or different person may also combine one part of a preparation of therapeutic agent A (again, either pre-formulated or newly formulated) with two parts of a preparation of therapeutic agent B (pre-formulated or newly formulated) provided that the drug formulations are at doses and quality approved for use in humans, and either administer the preparation to patient two (if authorized) or place the combined preparation in a vial labeled for patient two. Alternatively, that person may place in a vial labeled for patient two, an appropriate amount of a delivery vehicle formulation that contains therapeutic agents A and B co-encapsulated at a 1:2 ratio (either pre-formulated or formulated after identification of patient two's specific ratio), providing that the amount and quality of the formulation placed in the vial is approved for use in humans. The labeled vials may be stored, duplicated (for multiple administrations to the same patient), and/or transported under appropriate conditions prior to administration to patients. The labeled vials may be prepared in an authorized laboratory, clinic, pharmacy and the like.

In another embodiment of the invention, a kit is provided which comprises at least one pharmaceutical preparation containing at least one therapeutic agent and a second pharmaceutical preparation containing at least a second therapeutic agent. Using the previous example, a kit may be provided that comprises at least a pharmaceutical preparation of drug A and a pharmaceutical preparation of drug B. A person authorized may combine the appropriate amounts of each drug formulation (i.e., 1:5 of drugs A:B for patient one and 1:2 of drugs A:B for patient two) found in the kit prior to administration to patients by a qualified individual.

1. A method for determining a patient-specific, non-antagonistic ratio of two or more therapeutic agents comprising:
i) providing diseased cells obtained from a patient;
ii) characterizing a molecular phenotype of said diseased cells;
iii) matching the molecular phenotype of said diseased cells with the molecular phenotype of a cultured cell line;
iv) providing at least a first and a second therapeutic agent; and
v) assaying the first therapeutic agent in combination with the second therapeutic agent at various ratios in vitro on said cultured cell lines to determine a ratio of said first and second therapeutic agents that exhibits a non-antagonistic biological effect on said cultured cell lines,
whereby said ratio is identified as a patient-specific, non-antagonistic ratio.

2. The method of claim 1, wherein said patient-specific ratio exhibits said non-antagonistic biological effect on said cultured cell line over at least 5% of such concentration range where >1% of the cells are affected (fₐ>0.01) in said in vitro assay for biologic effect.

3. The method of claim 2, wherein said non-antagonistic biological effect is exhibited over at least 5% of the concentration range such that 20-80% of the cultured cells are affected (fₐ=0.2-0.8) in said in vitro assay.

4. The method of claim 3, wherein said non-antagonistic effect is exhibited over at least 20% of the concentration range such that 20-80% of the cultured cells are affected in said in vitro assay.

5. A method of preparing a patient-specific pharmaceutical preparation comprising:
i) providing a first composition comprising a first delivery vehicle, said first delivery vehicle having stably associated therewith a first therapeutic agent;
ii) providing a second composition comprising a second delivery vehicle, said delivery vehicle having stably associated therewith a second therapeutic agent; and
iii) combining said first composition and said second composition in a ratio of first therapeutic agent to second therapeutic agent that provides a non-antagonistic effect to cultured cells that have a molecular phenotype similar or identical to cells harvested from the diseased tissue or blood of said patient.

6. The method of claim 5, wherein combination of said first and second composition occurs immediately prior to use.

7. The method of claim 5, wherein said ratio exhibits said non-antagonistic biological effect on said cultured cells over at least 5% of such concentration range where >1% of the cells are affected (fₐ>0.01) in said in vitro assay for biologic effect.

8. The method of claim 7, wherein said non-antagonistic effect is exhibited over at least 5% of the concentration range such that 20-80% of the cultured cells are affected (fₐ=0.2-0.8) in said in vitro assay.

9. The method of claim 8, wherein said non-antagonistic effect is exhibited over at least 20% of the concentration range such that 20-80% of the cultured cells are affected in said in vitro assay.

10. A method of preparing a patient-specific pharmaceutical preparation comprising:
stably associating with a delivery vehicle at least a first and second therapeutic agent in a ratio of the first to second therapeutic agent that provides a non-antagonistic effect to cultured cells that have a molecular phenotype similar or identical to cells harvested from the diseased tissue or blood of the patient.

11. The method of claim 10, wherein the first therapeutic agent is stably associated with a first delivery vehicle and the second therapeutic agent is stably associated with a second delivery vehicle.

12. The method of claim 10, wherein said ratio exhibits said non-antagonistic biological effect on said cultured cells over at least 5% of such concentration range where >1% of the cells are affected (fₐ>0.01) in said in vitro assay for biologic effect.

13. The method of claim 12, wherein said non-antagonistic effect is exhibited over at least 5% of the concentration range such that 20-80% of the cultured cells are affected (fₐ=0.2-0.8) in said in vitro assay.

14. The method of claim 13, wherein said non-antagonistic effect is exhibited over at least 20% of the concentration range such that 20-80% of the cultured cells are affected in said in vitro assay.

15. The method of claim 10, wherein the first and second therapeutic agents are co-encapsulated in the same delivery vehicle.

16. The method of claim 10, wherein the first and second therapeutic agents are separately encapsulated with first and second delivery vehicles.

17. A composition prepared by the method of claim 5.

18. A composition prepared by the method of claim 10.

19. A method to provide a patient with an individualized treatment which method comprises administering to said patient the composition of claim 17.

20. A method to provide a patient with an individualized treatment which method comprises administering to said patient the composition of claim 18.

21. A method of providing a patient with an individualized treatment comprising:
administering to said patient a first composition comprising a first delivery vehicle associated with a first therapeutic agent and a second composition comprising a second delivery vehicle stably associated with a second therapeutic agent in a ratio that exhibits a non-antagonistic biological effect on cultured cells having a similar or identical molecular phenotype to cells obtained from said patient, wherein the pharmacokinetics of the first and second delivery vehicles are coordinated.

22. A method of providing a pharmaceutical preparation individualized to a particular patient comprising:
a) obtaining diseased cells from the patient;
b) characterizing the molecular phenotype of said patient's diseased cells;
c) matching the molecular phenotype of said patient's diseased cells with the molecular phenotype of cultured cells;
d) providing at least a first and a second therapeutic agent;
e) conducting an assay in vitro on said cultured cells to determine a ratio of at least a first and second therapeutic agent that exhibits a non-antagonistic biological effect on said cultured cells; whereby said ratio is determined as a patient-specific ratio for said individualized treatment; and
f) mixing a first composition comprising a first delivery vehicle associated with said first therapeutic agent with a second composition comprising a second delivery vehicle stably associated with said second therapeutic agent in the patient-specific ratio determined in e), wherein the pharmacokinetics of the delivery vehicles in said first and second compositions are coordinated.

23. The method of claim 22, wherein said ratio exhibits said non-antagonistic biological effect on said cultured cells over at least 5% of such concentration range where >1% of the cells are affected (fₐ>0.01) in said in vitro assay for biologic effect.

24. The method of claim 23, wherein said non-antagonistic effect is exhibited over at least 5% of the concentration range such that 20-80% of the cultured cells are affected (fₐ=0.2-0.8) in said in vitro assay.

25. The method of claim 24, wherein said non-antagonistic effect is exhibited over at least 20% of the concentration range such that 20-80% of the cultured cells are affected in said in vitro assay.
